(19) United States
(12) Patent Application Publication
(10) Pub. No.: US 2011/0152801 A1
(43) Pub. Date: Jun. 23, 2011

(54) Device for facilitating semen collection

(75) Inventors: Klaus Michael Andreas Vollrath, Mindarie (AU); Edward Joseph Khoury, Bateman (AU)

(73) Assignee: Klaus Michael Andreas Vollrath

(21) Appl. No.: 12/928,490

(22) Filed: Dec. 13, 2010

(30) Foreign Application Priority Data
Dec. 17, 2009 (AU) 2009906135
Nov. 19, 2010 (AU) 2010241535

(51) Int. Cl. A61M 1/00

(52) U.S. Cl. 604/322

(57) ABSTRACT

In a device to facilitate the collection of semen, a flange is provided for a semen cup, which extends inwardly from the wall of the cup and defines a central opening, thereby providing a peripheral flange to prevent semen from flowing back out and allowing the cup to be held in an inverted or angled orientation while providing the semen sample.

This application claims priority from Australian provisional patent application 2009906135 filed with the Australian Patent Office on Dec. 17, 2009, and from Australian non-provisional patent application 2010241535 filed with the Australian Patent Office on Nov. 19, 2010, citing as inventors: Klaus Michael Andreas Vollrath, and Edward Joseph Khoury.

### FIELD OF THE INVENTION

The invention relates to medical devices. In particular it relates to devices and methods for collecting semen samples.

### BACKGROUND OF THE INVENTION

It is not uncommon for couples wishing to have a baby to experience difficulties in conceiving. Fertility clinics exist for the very purpose of assisting people in determining the cause of the problem and, if possible, taking steps toward addressing some of the hurdles.

This typically involves the collection of semen samples for further lab testing and analysis. However, one of the difficulties commonly experienced by patients in providing the semen sample is getting the sample into the container or cup.

A typical cup for collecting semen samples is shown in FIG. 1, and comprises a plastic cup 100 with an upper threaded portion 102 for receiving a complementarily threaded cap 104 for closing the open end 110. However, an erect penis is typically angled upwardly, thus requiring the cup 100 with its open end 110 to be essentially held upside down or at least partially inverted. This makes the task of ejaculating into the cup, while avoiding the ejaculate from flowing back out of the cup, a difficult and cumbersome process. Patients therefore often fail to provide a full ejaculate.

During the process of providing a semen sample, the patient also runs the risk of touching the inner wall 112 of the cup 100 with his penis, thereby exposing the semen sample to contaminants such as bacteria and lubricants.

The present invention seeks to address this problem by providing a new ejaculate capturing device.

### SUMMARY OF THE INVENTION

According to the invention there is provided an insert for a semen cup, wherein the semen cup includes an open end and a peripheral cup wall with an inner surface and an outer surface, the insert comprising a ring-shaped structure that includes a peripherally extending outer surface formed to complementarily engage the inner surface of the peripheral cup wall, and further comprising an inwardly extending flange that defines a penis-engaging surface with a central opening. The penis-engaging surface preferably has a convex, concave or conical portion.

For purposes of this application the terms “wall” and “surface” will be used in the singular to cover not only circumferentially extending walls but also multi-faceted walls with more than one wall section.

The peripheral outer surface of the insert may be defined by an outer surface of a peripheral insert wall, the insert wall having an upper end and a lower edge. The flange may extend inwardly from the upper end of the insert wall. The insert may include one or more outwardly extending tabs or a continuous outwardly extending lip engageable with an upper edge of the peripheral cup wall to avoid the insert being pushed too far into the cup.

The outer surface of the insert wall, or the peripheral flange may include one or more breather channels for providing air flow communication between the inside of the cup and the outside when the insert is inserted into the cup.

Further, according to the invention there is provided a cap for a cup, comprising a central body, and a peripheral wall with a threaded inner surface, wherein the central body defines an inner surface, the cap further comprising at least one finger extending from the inner surface of the central body and having a hook at a free end of the finger. The cap may include a peripheral flange arranged concentrically with the peripheral wall, the peripheral flange defining an outwardly facing sealing surface for engaging an inner surface of the cup.

Still further, according to the invention, there is provided an adaptor for a semen cup, wherein the semen cup includes an open end and a peripheral cup wall that is provided with an external thread toward the open end, the adaptor comprising a ring-shaped structure that includes a peripherally extending adaptor wall with an internal thread configured to complementarily engage the external thread of the cup wall, and further comprising an inwardly extending flange that defines a penis-engaging surface with a central opening. The penis-engaging surface preferably has a convex, concave or conical portion.

Still further, according to the invention, there is provided a semen cup that includes a base defining a lower end of the cup, and a peripheral cup wall extending from the base to define an upper end, the cup further including an inwardly extending peripheral flange that extends inwardly from or near the upper end of the cup and defines a penis-engaging outer surface with a central opening. The penis-engaging outer surface preferably has a convex, concave, or conical configuration. The peripheral flange may include one or more breather channels extending through the peripheral flange between an inside and an outside of the cup.

### BRIEF DESCRIPTION OF THE DRAWINGS

FIG. 1 shows a three dimensional view of a prior art semen cup,

FIG. 2 shows a three dimensional view of one embodiment of an insert of the invention;

FIG. 3 shows a three dimensional view of another embodiment of an insert of the invention, showing the upper or outer side of the insert;

FIG. 4 shows a three dimensional view of the embodiment of FIG. 3, showing the lower or inner side of the insert;

FIG. 5 shows a bottom view of the embodiment of FIG. 3;

FIG. 6 shows a sectional view along A-A of FIG. 5;

FIG. 7 shows a three dimensional view of a semen cup with an insert, of one embodiment of the invention and a screw cap;

FIG. 8 is a sectional side view of a cup with an insert of the invention, showing the positioning of the penis in relation to the cup and insert;

FIG. 9 is a sectional side view of the cup and insert of FIG. 8, showing the positioning of the penis positioned against the insert;

FIGS. 10-12 show sectional side views of a semen cup arrangement of the invention in different orientations during sampling of an ejaculate,

FIG. 13 shows a sectional side view of a cup with an insert of the invention and the cup partially closed by a cap;

FIG. 14 shows a sectional side view of the cup and insert of FIG. 13 with the cap fully engaged with the cup;

FIG. 15 shows a sectional side view of another embodiment of the invention making use of a modified cap;

FIG. 16 shows a three dimensional view of yet another embodiment of the invention usable with a conventional cup, wherein an adaptor engageable with the threads of the cup is used instead of an insert;

FIG. 17 shows a sectional side view of yet another embodiment of the invention making use of an insert and a modified cap;

FIG. 18 shows a sectional side view of yet another embodiment of an insert and modified cap of the invention;

FIG. 19 shows a three dimensional view of yet another embodiment of an insert of the invention, and

FIG. 20 shows a three dimensional view of one embodiment of a semen cup in accordance with the invention.

### DETAILED DESCRIPTION OF THE INVENTION

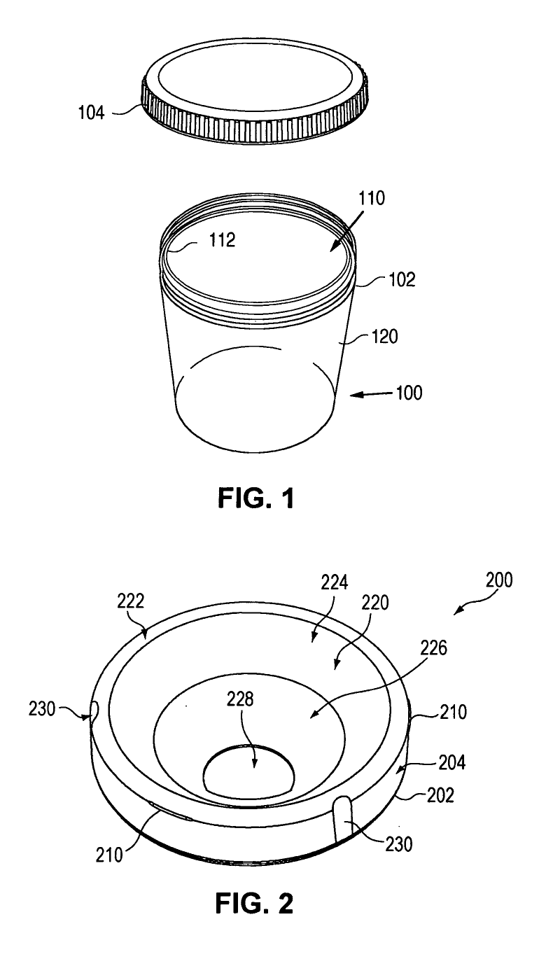

One embodiment of the invention is shown in FIG. 2, which comprises an insert 200 for insertion into a prior art semen cup. It will be appreciated that the insert can be manufactured in different sizes and can be dimensioned appropriately to fit into any standard semen cup. The insert 200 has a ring-shaped configuration with a peripheral wall 202 having an outer surface 204 that is shaped and sized to fit into the open end of a semen cup (such as the open end 110 of cup 100 shown in FIG. 1) to complementarily engage the inner surface 112 of the cup 100 at the open end. In the cup shown in FIG. 1, the cup's peripheral wall 120 is beveled to give the cup a frusto-conical shape. Therefore, it will be appreciated that the insert 200 will be wedged in place against the inner surface of the cup's peripheral wall 120 as it is inserted deeper into the cup. However, insofar as the cup or insert are made of a flexible material such as a plastics material, the insert will typically tend to be pushed downward into the cup during use. In order to ensure that the insert 200 remains near the top of the cup, tabs 210 are included that extend outwardly from the outer surface 204 of the wall 202, at the upper end of the wall 202.

As shown in FIG. 2, an inwardly extending ledge or flange 220 extends inwardly from the upper edge of the peripheral wall 202. In this embodiment the ledge or flange 220 includes a first, outermost section in the form of a slightly rounded section 222, a concave section 224, and a convex section 226 having a convex outer surface for engaging the penis. These sections are best seen in the embodiment of FIG. 6, which has a similar set of sections for the flange 220. The convex section 226 defines a central hole 228 through which the ejaculate is deposited into the semen cup. Since the penis will typically engage the outer surface of the concave section 226 in a substantially air-tight seal the insert 200 in this embodiment is provided with air vents in the form of channels 230 formed in the insert's peripheral wall 202 to allow air to pass out of the cup as the ejaculate enters the cup.

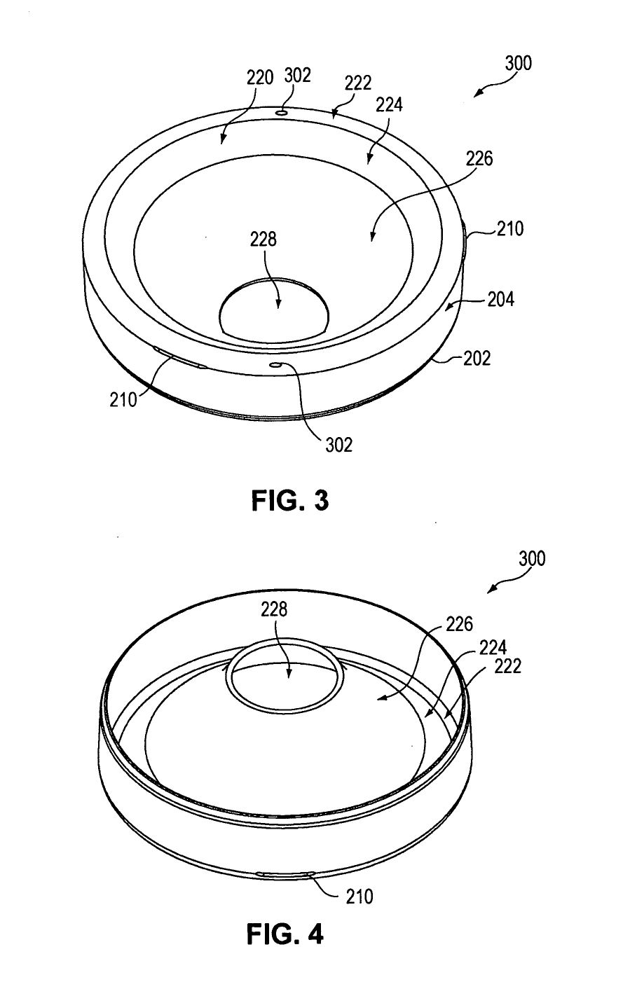

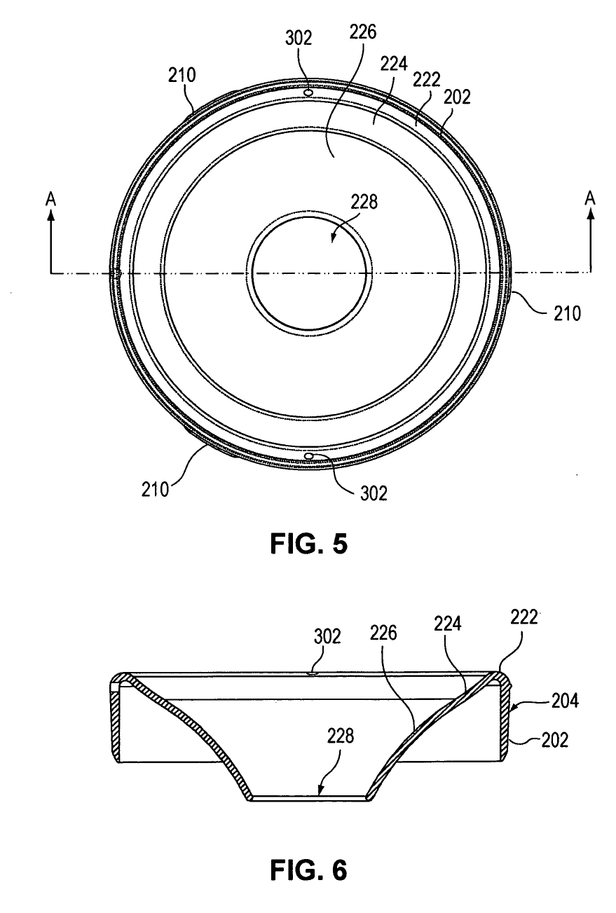

Another embodiment 300 of the invention is shown in FIGS. 3-6, which instead of air channels 230, is provided with holes 302 for venting air during use. Since this embodiment is otherwise similar to that of FIG. 2, similar elements of the insert are depicted by the same reference numerals. FIG. 3 shows the insert in three dimensions from the top, while FIG. 4 shows the insert in three dimensions from below.

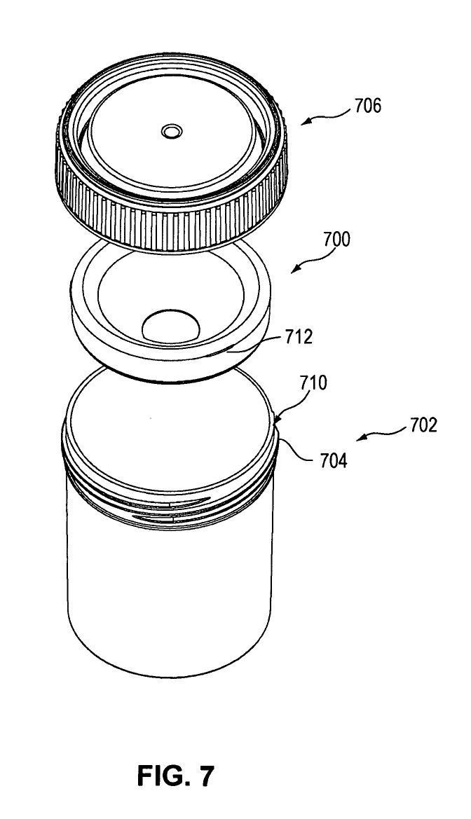

In the preferred embodiment the insert of the invention is dimensioned to fit into a standard semen cup, thereby allowing a standard closure to be used with the cup once the sample has been provided. FIG. 7 shows an insert 700 of the invention in relation to a typical semen cup 702 as known in the art. The cup 702 is provided with at thread 704 at its upper outer surface to receive a complementarily threaded cap 706 as known in the art. During use, i.e., for purposes of collecting semen, the insert 700 is inserted into the cup 702 and is held in place along the upper rim 710 of the cup by tabs 712 that extend from the upper end of the peripheral wall of the insert as was discussed above.

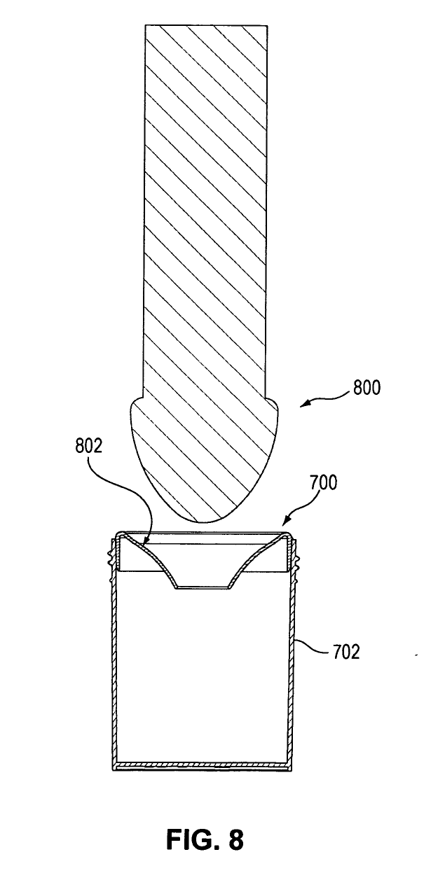

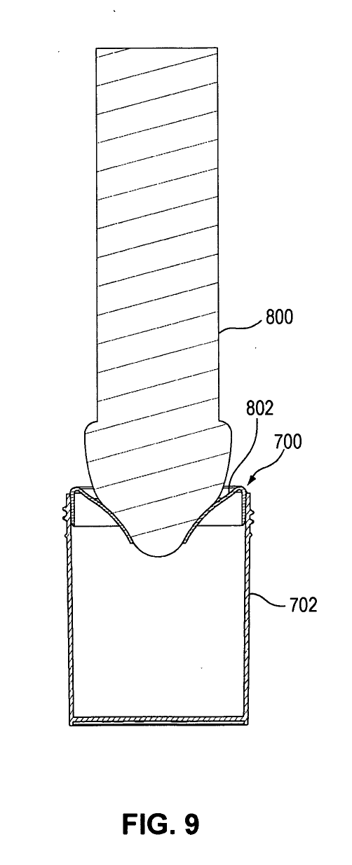

FIG. 8 shows the cup 702 with the insert 700 inserted into the cup for purposes of engaging the penis 800 with the upper our outer surface of the flange 802. This is best illustrated in the sectional side view of FIG. 9, which shows the penis 800 engaging mainly the convex portion of the flange 802.

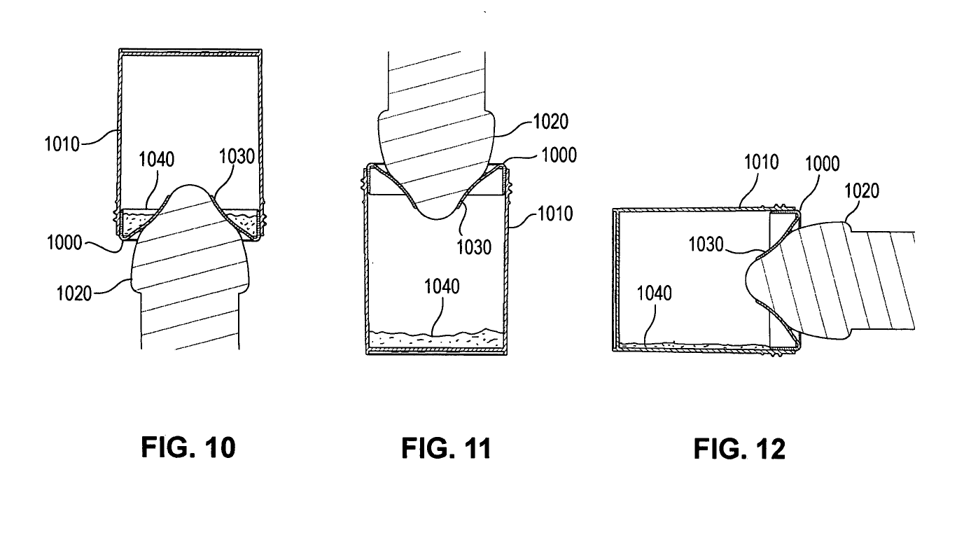

FIGS. 10-12 show an insert 1000 of the invention inserted into a standard semen cup 1010 for use in gathering a semen sample. FIG. 10 shows the cup 1010 held in an inverted position with the opening of the cup and the insert 1000 toward the bottom. This positioning of the cup allows the collection of semen with the penis 1020 in a more natural, upwardly pointing orientation. The inwardly extending flange 1030 acts as a dam wall for retaining the semen 1040 inside the cup. As shown in FIG. 11, the cup 1010 can be held in a conventional, substantially vertical position with the opening at the top. In this position the insert 1000 nevertheless plays an important role in that it keeps the penis 1020 and any contaminants away from the inner surface of the cup 1010. The cup may also be held in other orientations to accommodate the patient, as illustrated in FIG. 12, which shows the cup in a horizontal orientation.

It will therefore be appreciated that in the orientation shown in FIGS. 10 and 12 the insert 1000 not only keeps contaminants away from the inside of the cup but provides the additional benefit of retaining the ejaculate in the cup since the flange 1030 acts as a barrier or ledge preventing the ejaculate from flowing out.

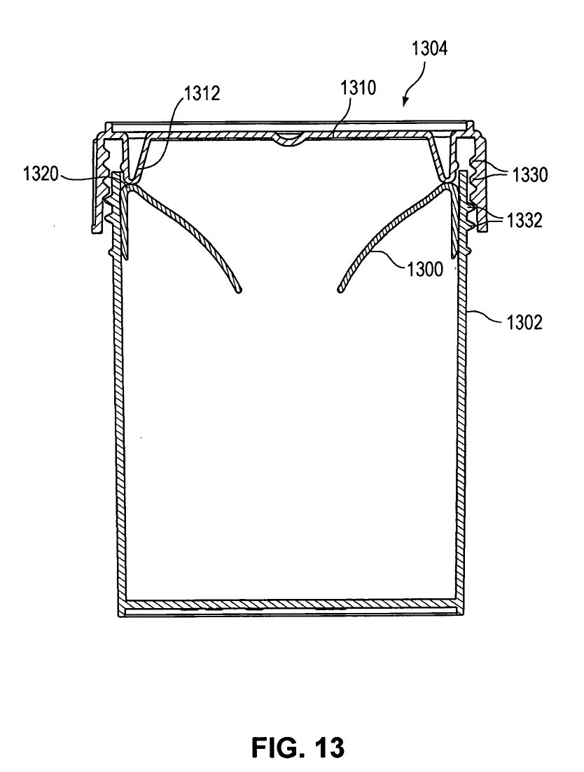

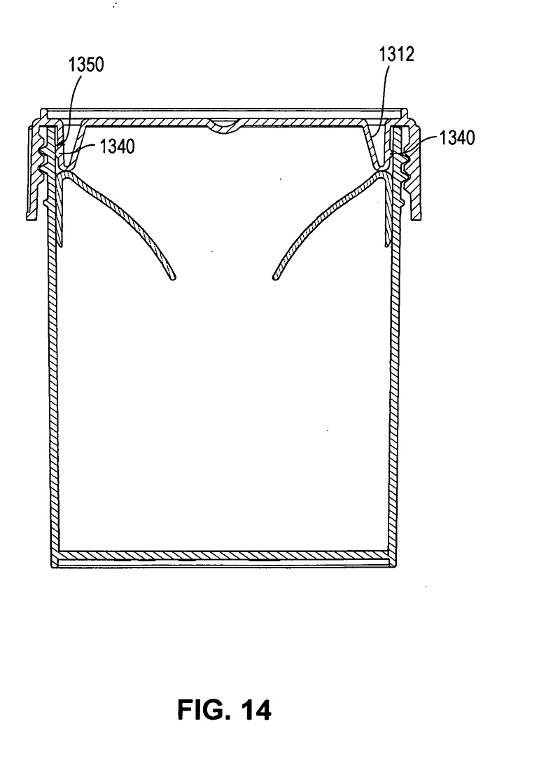

Once the semen has been deposited in the cup the insert can either be removed or remain in the cup when the cap is screwed onto the cup. In the embodiment shown in FIG. 13, the insert 1300 remains in the cup 1302 when the cap 1304 is placed on the cup. As shown in FIG. 13, the cap 1304 has an inner wall 1310, which has a downwardly extending peripheral flange 1312 that seals with the inner surface of the side wall of the cup 1302. In this embodiment the sealing flange 1312 of the cap 1304 impinges upon the rounded upper portion 1320 of the inwardly extending flange of the insert 1300 before the threads 1330 of the cap have an opportunity to engage the complementary threads 1332 of the cup. Thus, as shown in FIG. 13, the insert first has to be pressed further into the cap to allow the threads 1330 to catch the threads 1332, whereafter the cap 1304 can be screwed onto the cup 1302, thereby further pressing the insert 1300 downwardly into the cup, as shown in FIG. 14. The peripheral flange 1312 of the cap is provided with a peripherally extending rib 1340 which acts as a seal against the inner surface 1350 of the cup wall.

It will be appreciated that the tabs 210 (FIG. 2) are chosen to be small enough to allow the insert to be pushed into the cup when sufficient pressure is exerted on the insert, while acting retaining the insert at the top of the cup during use (ejaculation) by the patient.

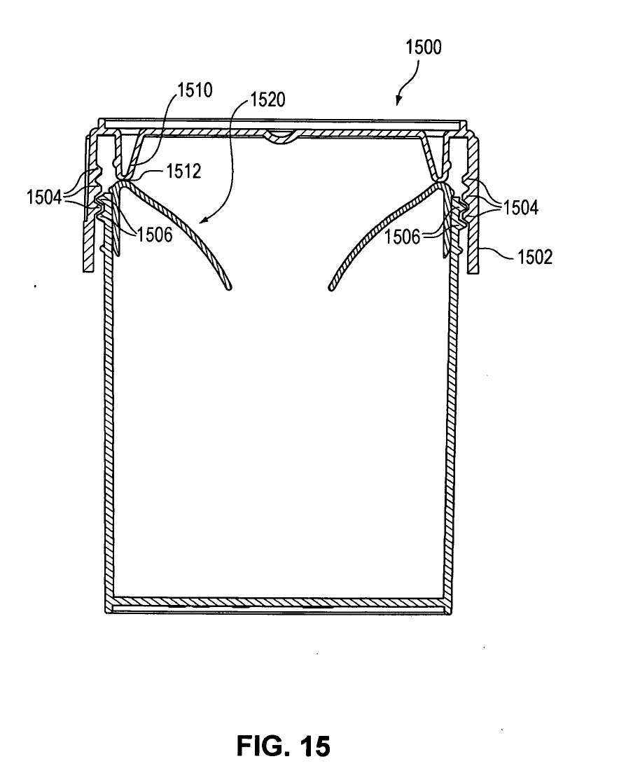

In order to avoid the two step process in closing the cup after use, namely first pressing down on the cap to push the insert down far enough to allow the threads on the cap and cup to engage one another, and then screwing the cap down, another embodiment makes use of a cap with a longer peripheral wall. Such an embodiment is shown in FIG. 15, in which the peripheral wall 1502 of the cap 1500 is made longer to allow the threads 1504 on the inner surface of the peripheral wall 1502 to be positioned lower on the cap and thereby allow the threads 1504 to engage the threads 1506 of the cap before the flange 1510 on the cap impinges upon the upper rounded section 1512 of the insert 1520. When the cap is thereafter screwed downward onto the cup, the flange 1510 presses the insert 1520 downward into the cup.

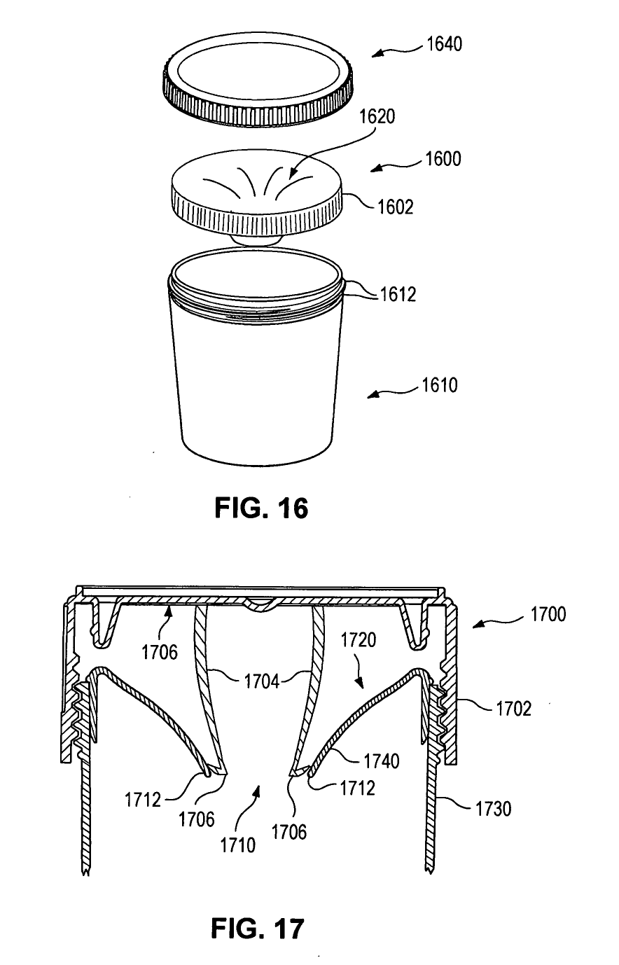

In yet another embodiment the penis-engaging adaptor is implemented not as an insert as in the above embodiments but as a separate screw-on adaptor 1600 as shown in FIG. 16. In this embodiment the peripheral wall 1602 of the adaptor 1600 is provided with an inner thread (not visible) for engaging the threads 1612 on the cup 1610. The inwardly extending flange 1620 on the adaptor 1600 is similar to the flange 220, 802 described in the above embodiments. This embodiment, however, has the benefit that the user can remove the adaptor 1600 after the ejaculate has been deposited in the cup, thereby allowing the regular cap 1640 to be screwed onto the cup without interference by an insert. This embodiment also has the benefit that lab technicians dealing with the semen sample have easier access to the semen and are not hampered by an insert in the cup.

In yet another embodiment, shown in FIG. 17, the regular cap is replaced with a cap similar to that described with respect to FIG. 15. However, in this embodiment the cap 1700, in addition to the longer peripheral wall 1702 is provided with four flexible fingers 1704 extending downwardly from the inner surface 1706 of the cap. The fingers 1704 are positioned so that there lower extremities are spaced further apart than the diameter of the central opening 1710 of the insert 1720. The fingers 1704 are also provided with outwardly extending hooks 1714. Thus, when the cap 1700 is screwed down on the cup 1730, the central hooks move downward, being forced inward by the slanted surface of the flange 1740. Once the hooks 1706 pass through the opening 1710 they spring back underneath the lower edge 1712 of the insert 1720. Thus when the cap 1700 is subsequently unscrewed by lab personnel the hooks 1706 engage the lower edge 1712 of the insert 1720 and extract the insert 1720 from the cup to allow the lab personnel to access the semen sample more easily. It will be appreciated that as few as one hooked finger could be provided to extract the insert. The finger can also have different configurations. For example the finger could comprise a central post with multiple hooks extending from the post, wherein the hooks are deformable or pivotable in one direction only to facilitate insertion through the central opening of the insert, but resisting deformation or pivoting in the opposite direction, thereby allowing the cap with its post and hooks to extract the insert when the cap is unscrewed.

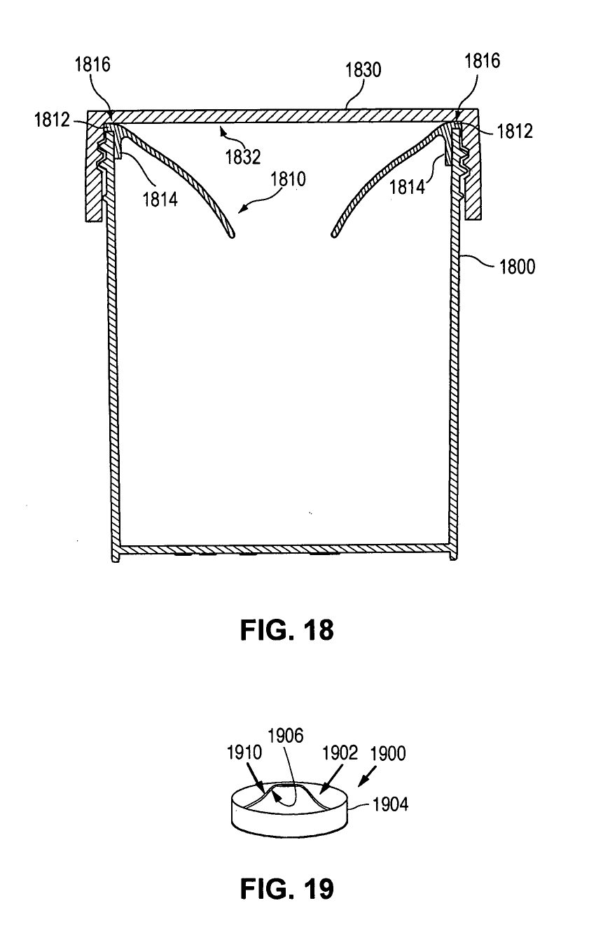

In yet another embodiment of an insert of the invention, instead of making use of tabs e.g. tabs 210 in the FIG. 2 embodiment, a retaining lip is provided that extends around the entire periphery of the insert and engages the upper rim of the cup. Such an embodiment is shown in FIG. 18, which shows a conventional cup 1800 with an insert 1810 of the invention inserted into the cup 1800. As is shown in FIG. 18, a lip 1812 extends from the side wall 1814 of the insert 1810 and rests on top of the rim of the cup 1800 when the insert is inserted into the cup. The cap 1830, which replaces the conventional cap described with respect to FIGS. 13-15, is provided without the downwardly extending flange 1312, 1510 that was seen in the embodiments of FIGS. 13-15. Thus the cap does not force the insert into the cup when the cap engages the cup. The cap, instead seals by engaging the inner surface 1832 of the cap 1830 with the upper surface 1816 of the lip 1812. This, arguably, does not provide as good a seal as the embodiments of FIGS. 13-15 but is adequate for cups that are not exposed to the pressures of vacuum powered courier pipes that are used in some laboratories.

In the embodiments discussed above the peripheral flange of the insert or adaptor e.g., flange 220 included 3 sections, however it will be appreciated that the inner two sections could define a smooth outer surface, preferably having a conical, concave or convex shape. FIG. 19 shows an embodiment of an insert 1900 in which the flange 1902 extends inwardly from the peripheral wall 1904 in one continuous section having a smooth, convex outer surface 1906. It will be appreciated that the insert 1900 shown in FIG. 19 is upside-down, therefore the upper surface 1910 in this orientation is the inner surface of the flange 1902, whereas the lower surface 1906 in this orientation is the outer surface.

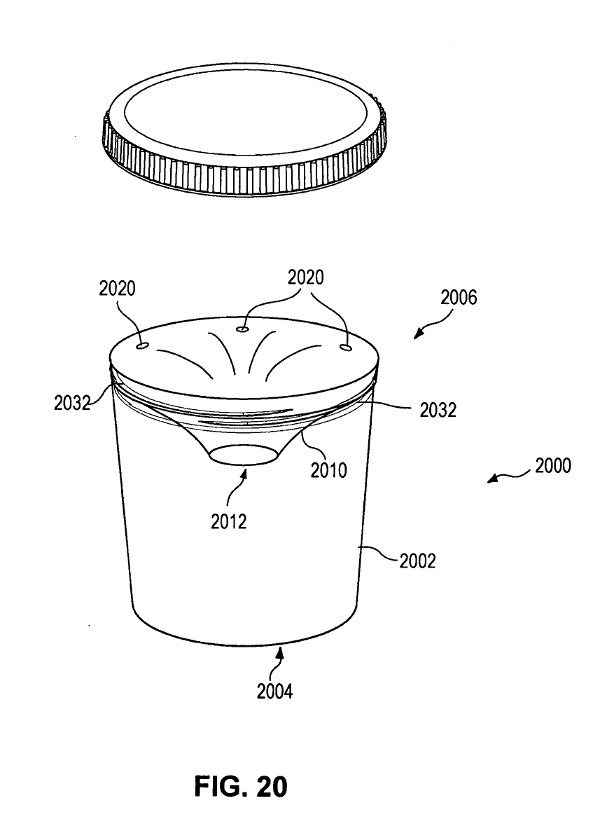

The embodiments discussed above showed the device for semen collection implemented as an insert or separate adaptor for an existing semen cup. It will be appreciated that the invention could be implemented as a new type of semen cup with built-in flange similar to the flanges 220, 1902 discussed above for the insert or adaptor. One such embodiment is shown in FIG. 20. The semen cup 2000 of this embodiment is made of plastics material and is configured to be similar in size and shape to a standard prior art semen cup such as the cup shown in FIG. 1. It will be appreciated that other materials such as glass can be used in other embodiments. Similar to the prior art cup 100, the cup 2000 of the present invention includes side walls 2002, a flat, closed bottom end 2004, and an upper end 2006. The cup 2000, however, distinguishes itself from the prior art cup in that it includes an inwardly extending flange 2010 with a central opening 2012. In this embodiment the flange 2010 is shown to have a slightly convex outer surface for engaging the penis during use. Air channels in the form of holes 2020 are provided in the flange 2010 to allow air to escape from the inside of the cup 2000 as semen is ejaculated into the cup. A cap 2030 is provided with internal threads (not shown) for engaging complementary threads 2032 on the outer surface of the cup. The cap 2030 is sized and shaped to correspond to existing prior art caps for easier handling, as is discussed below.

It will be appreciated that there is a benefit to sizing the inserts of the invention for use in existing semen cups or integrating the flange into cups that have a similar size to existing semen cups since laboratories that test the sample may use handling tools that are set up for existing cup sizes. For example, some laboratories use vacuum powered courier pipes to send samples from one part of the laboratory to the other, therefore in such cases the sample cups must preferably be able to withstand the pressure requirements of the vacuum powered courier pipe and also conform to the size and specifications of the courier pipe.

The size of the central opening, such as opening 228 in the embodiment of FIG. 2 or the opening 2012 in the FIG. 20 embodiment is also important. The central opening in the flange is preferably sized to allow only the urethral opening of the penis or a small region surrounding the urethral opening to fill the central opening of the flange. This ensures the sterility of the semen sample and keeps any contaminants on the outside. The opening in the flange also allows a sample pipette to easily pass through and reach into the corners to suction out the semen sample.

While the invention has been described with respect to a few specific embodiments it will be appreciated that the invention can be implemented in different ways without departing from the scope of the invention as defined by the claims.

What is claimed is:

1. An insert for a semen cup, wherein the semen cup includes an open end and a peripheral cup wall with an inner surface and an outer surface, the insert comprising
a peripherally extending outer surface configured to complementarily engage the inner surface of the peripheral cup wall, and
an inwardly extending flange that defines a penis-engaging surface with a central opening.

2. An insert of claim 1, wherein the penis-engaging surface has a convex, concave or conical portion.

3. An insert of claim 2, wherein the peripheral outer surface of the insert is defined by an outer surface of a peripheral insert wall, the insert wall having an upper end and a lower end.

4. An insert of claim 3, wherein the flange extends inwardly from the upper end of the insert wall.

5. An insert of claim 4, wherein the insert includes one or more outwardly extending tabs or an outwardly extending peripheral lip engageable with an upper edge of the peripheral cup wall.

6. An insert of claim 3, wherein at least one of the outer surface of the insert wall, and the peripheral flange includes one or more breather channels for providing air flow communication between the inside of the cup and the outside when the insert is inserted into the cup.

7. An insert of claim 5, wherein at least one of the outer surface of the insert wall, and the peripheral flange includes one or more breather channels for providing air flow communication between the inside of the cup and the outside when the insert is inserted into the cup.

8. A cap for a cup, comprising
a central body, and
a peripheral wall with a threaded inner surface, wherein the central body defines an inner surface, the cap further comprising
at least one finger extending from the inner surface of the central body and having a hook at a free end of the finger.

9. A cap of claim 8, wherein at least part of the at least one finger is flexible.

10. A cap of claim 9, further comprising a peripheral flange arranged concentrically with the peripheral wall, the peripheral flange defining an outwardly facing sealing surface for engaging an inner surface of the cup.

11. An adaptor for a semen cup, wherein the semen cup includes an open end and a peripheral cup wall that is provided with an external thread toward the open end, the adaptor comprising a ring-shaped structure that includes a peripherally extending adaptor wall with an internal thread configured to complementarily engage the external thread of the cup wall, and an inwardly extending flange that defines a penis-engaging surface with a central opening.

12. An adaptor of claim 11, wherein the penis-engaging surface has a convex, concave or conical portion.

13. A semen cup, comprising
a base defining a lower end of the cup,
a peripheral cup wall extending from the base to define an upper end of the cup, and
an inwardly extending peripheral flange that extends inwardly from or near the upper end of the cup and defines a penis-engaging outer surface with a central opening.

14. A semen cup of claim 8, wherein the penis-engaging outer surface has a convex, concave, or conical configuration.

15. A semen cup of claim 9, wherein the peripheral flange includes one or more breather channels extending through the peripheral flange between an inside and an outside of the cup.
